(19) United States
(12) Patent Application Publication
(10) Pub. No.: US 2017/0165239 A1
(43) Pub. Date: Jun. 15, 2017

(54) USE OF BETA-CARBOLINE ALKALOID IN INHIBITING XANTHINE OXIDASE ACTIVITY

(71) Applicant: Food Industry Research And Development Institute, Hsinchu City (TW)

(72) Inventors: Ming-Der Wu, Hsinchu City (TW); Ming-Jen Cheng, Hsinchu City (TW); Yen-Lin Chen, Hsinchu City (TW); Siao-Jhen Chen, Tainan City (TW); Li-Wen Yu, Hsinchu City (TW); Hsun-Yin Hsu, Hsinchu City (TW)

(21) Appl. No.: 15/370,527

(22) Filed: Dec. 6, 2016

Related U.S. Application Data
(60) Provisional application No. 62/264,933, filed on Dec. 9, 2015.

(51) Int. Cl. A61K 31/437; C12Q 1/26; A61K 9/00

(52) U.S. Cl. CPC A61K 31/437; A61K 9/0053; C12Y 117/03002; C12Q 1/26; G01N 2333/9029

(57) ABSTRACT

A method for inhibiting xanthine oxidase and for reducing uric acid levels using a beta-carboline alkaloid compound of formula (I), wherein R₁ is selected from the group consisting of a carboxyl group, a carboxylate group, a carboxamide group and hydrogen, and R₂ is selected from the group consisting of —CH₂COOCH₃, a methoxy group, hydrogen and a methyl group. Also disclosed is a method for monitoring xanthine oxidase inhibiting activity level.

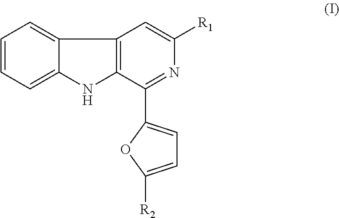

### CROSS-REFERENCE TO RELATED APPLICATIONS

The present application claims the benefit of U.S. Provisional Patent Application No. 62/264,933, filed on Dec. 9, 2015, which is hereby incorporated by reference.

### BACKGROUND

Field of the Invention

The invention relates to methods for inhibiting xanthine oxidase activity in a subject in need thereof, methods for reducing uric acid levels in a subject in need thereof, and methods for monitoring xanthine oxidase inhibiting activity level.

Background Information

Uric acid is the end product of purine metabolism in the body. A high level of uric acid in the blood leads to the formation and deposition of uric acid crystals in the joints, kidneys, and other organs. A blood uric acid concentration higher than 7 mg/dL is considered to be hyperuricemia. Hyperuricemia is a common metabolic disorder that is associated with gout, hypertension, cardiovascular disease, diabetes, and kidney disease.

Xanthine oxidase is a key enzyme in the synthesis of uric acid. As a result, inhibition of xanthine oxidase activity can reduce the production of uric acid. Indeed, the xanthine oxidase inhibitor (XOI), uricase, is effective for lowering the concentration of uric acid in the blood. Uricase is an enzyme not found in humans. It is typically isolated as a recombinant mammalian protein and administered by IV infusion. As such, it can be expensive to produce and difficult to administer.

Two xanthine oxidase inhibitors are administered clinically to lower serum uric acid levels, allopurinol and febuxostat. However, each have side effects, such as allergic reactions, gastrointestinal discomfort, leukopenia and thrombocytopenia, hepatitis, nephropathy, and 6-mercaptopurine toxicity, which in certain cases can lead to death. Accordingly, there are unmet needs in the market for safe and effective xanthine oxidase inhibitors.

A compound named Flazin, a beta-carboline alkaloid, was isolated in 1936, and its structure was characterized in 1986. Flazin has also found in Japanese rice vinegar, soy sauce and miso (Shin-ichi Nadatsuka et al., Tetrahedron Letters 27(29):3399-3402). Flazin was shown to have anti-tumor activity in 2002, and it is also known for its anti-HIV activities (Su B N et al., Planta Med. 68(8):730-733), and for its ability to inhibit superoxide anion generation (Yang M L et al., J Nat Prod. 74(9):1996-2000).

### SUMMARY OF THE INVENTION

The present disclosure provides, amongst others, methods of inhibiting xanthine oxidase activity, or reducing uric acid levels, using beta-carboline alkaloid compounds.

In some embodiments, methods for inhibiting xanthine oxidase activity in a subject in need thereof are provided, the methods comprising the step of contacting a composition comprising, or consisting essentially of, a beta-carboline alkaloid compound of formula (I), with the xanthine oxidase. In these methods, R₁ of formula (I) is selected from the group consisting of a carboxyl group, a carboxylate group, a carboxamide group and hydrogen, and R₂ of formula (I) is selected from the group consisting of —CH₂COOCH₃, a methoxy group, hydrogen and a methyl group.

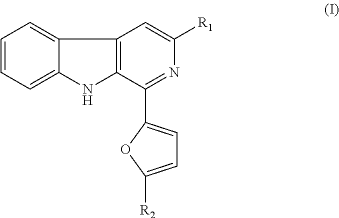

Also provided by the present disclosure are methods for reducing uric acid levels in a subject in need thereof, the methods comprising the step of administering to a subject an effective amount of a beta-carboline alkaloid compound of formula (I). In these methods, R₁ of formula (I) is selected from the group consisting of a carboxyl group, a carboxylate group, a carboxamide group and hydrogen, and R₂ of formula (I) is selected from the group consisting of —CH₂COOCH₃, a methoxy group, hydrogen and a methyl group.

Additionally, methods for monitoring xanthine oxidase inhibition activity are also provided by the present disclosure, these methods comprising the steps of (a) determining an amount of a beta-carboline alkaloid compound of formula (I) that correlates with a desirable level of xanthine oxidase inhibition activity (hereinafter termed “a desirable amount”); (b) measuring the amount of said beta-carboline alkaloid compound in a batch of a composition to be monitored; and (c) determining that said batch has reached said desirable xanthine oxidase inhibition activity when said amount is equal to or higher than the desirable amount. In these methods, R₁ in formula (I) is selected from the group consisting of a carboxyl group, a carboxylate group, a carboxamide group and hydrogen, and R₂ is selected from the group consisting of —CH₂COOCH₃, a methoxy group, hydrogen and a methyl group.

The details of one or more embodiments of the invention are set forth in the description, in the drawings, and in the examples below. Other features, objects, and advantages of the invention will be apparent from the detailed description of several embodiments and also from the claims. All publications and patent documents cited herein are incorporated by reference in their entirety.

### BRIEF DESCRIPTION OF THE DRAWINGS

FIG. 1 is a graph showing xanthine oxidase inhibitory activity of a beta-carboline alkaloid compound, Flazin.

FIG. 2 is a graph showing the Flazin concentration and xanthine oxidase inhibitory activity of the 9 samples.

FIG. 3 is a graph showing the correlation between Flazin concentration and xanthine oxidase inhibitory activity.

### DETAILED DESCRIPTION

As set forth above, methods for inhibiting xanthine oxidase activity are disclosed herein, these methods comprising the step of contacting a composition comprising, or consisting essentially of, a beta-carboline alkaloid compound of formula (I), with the xanthine oxidase,

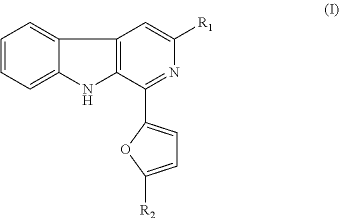

wherein R₁ is selected from the group consisting of a carboxyl group, a carboxylate group, a carboxamide group and hydrogen, and R₂ is selected from the group consisting of —CH₂COOCH₃, a methoxy group, hydrogen and a methyl group. In some embodiments of the compound of formula (I), R₁ is selected from the group consisting of a carboxyl group and hydrogen. In other embodiments, R₁ is hydrogen, and R₂ is hydrogen. In one particular embodiment, the compound of formula (I) is 1-[5-(hydroxymethyl)furan-2-yl]-9H-pyrido[3,4-b]indole-3-carboxylic acid, and/or 1-(5-hydroxymethyl-2-furyl)-β-carboline-3-carboxylic acid. In other embodiments, the beta-carboline alkaloid compound of formula (I) can be derived from flazin by the methods disclosed in J. G. Tang et al., Chemistry & Biodiversity 5:447-460 or Y. H. Wang et al., Biochemical and Biophysical Research Communications 355:1091-1095.

In some embodiments of these methods, the contacting step can be performed in vitro. For example, a preparation of xanthine oxidase can be placed in a vessel together with the compound. In other embodiments, the contacting step is accomplished by administering the composition orally to a subject having xanthine oxidase, or to a subject in need of xanthine oxidase inhibition.

In some embodiments, the beta-carboline alkaloid compound of formula (I) can be present in a fermentation product. The fermentation product can be purified or unpurified; the fermentation product can be highly purified, or only slightly purified, such as by using liquid-solid separation.

Methods for reducing uric acid levels in a subject in need thereof are also provided by the present disclosure, these methods comprising the step of administering to the subject an effective amount of a beta-carboline alkaloid compound of formula (I), wherein R₁ is selected from the group consisting of a carboxyl group, a carboxylate group, a carboxamide group and hydrogen, and R₂ is selected from the group consisting of —CH₂COOCH₃, a methoxy group, hydrogen and a methyl group. In some embodiments of the compound of formula (I), R₁ is selected from the group consisting of a carboxyl group and hydrogen. In other embodiments, R₁ is hydrogen, and R₂ is hydrogen. In one particular embodiment, the compound of formula (I) is 1-[5-(hydroxymethyl)furan-2-yl]-9H-pyrido[3,4-b]indole-3-carboxylic acid, and/or 1-(5-hydroxymethyl-2-furyl)-β-carboline-3-carboxylic acid.

In some embodiments, the subject is hyperuricemic. In other embodiments, the subject suffers from gout.

In some embodiments, the compound is administered orally, however, other routes of administration are contemplated herein, including, for example intravenous administration, intramuscular administration, intraperitoneal administration or subcutaneous injection.

In some embodiments, the compound is administered as the only active agent. In other embodiments, additional active agents are included. These additional active agents can include, for example, other xanthine oxidase inhibitors, such as allopurinol and febuxostat. In some embodiments, the beta-carboline alkaloid compound of formula (I) can be present in a fermentation product. The fermentation product may be purified or unpurified; the fermentation product can be highly purified, or only slightly purified, such as by using liquid-solid separation.

The amount of the compound administered is effective for reducing uric acid levels in the subject. A skilled artisan can easily determine the effective amount by, e.g., measuring changes in the concentration of uric acid in the blood of the subject. In some embodiments, the effective amount of the compound is 200-400 mg/kg body weight.

Additionally, methods for monitoring xanthine oxidase inhibition activity are provided by the present disclosure, these methods comprising the steps of (a) determining an amount of a beta-carboline alkaloid compound of formula (I) that correlates with a desirable level of xanthine oxidase inhibition activity (“a desirable amount”); (b) measuring the amount of said beta-carboline alkaloid compound in a batch of a composition to be monitored; and (c) determining that said batch has reached said desirable xanthine oxidase inhibition activity when said amount is equal to or higher than the desirable amount. In some embodiments of these methods, R₁ of Formula (I) is selected from the group consisting of a carboxyl group, a carboxylate group, a carboxamide group and hydrogen, and R₂ of Formula (I) is selected from the group consisting of —CH₂COOCH₃, a methoxy group, hydrogen and a methyl group. In other embodiments, R₁ is selected from the group consisting of a carboxyl group and hydrogen. In other embodiments, R₁ is hydrogen, and R₂ is hydrogen. In one particular embodiment, the compound of formula (I) is 1-[5-(hydroxymethyl)furan-2-yl]-9H-pyrido[3,4-b]indole-3-carboxylic acid, and/or 1-(5-hydroxymethyl-2-furyl)-β-carboline-3-carboxylic acid.

The amount of the beta-carboline alkaloid compound can be measured by a variety of suitable techniques that are well known in the art. For example, the amount of said beta-carboline alkaloid compound can be measured using high performance liquid is chromatography (HPLC). In one particular embodiment, the HPLC analysis conditions for measuring Flazin are as follows:

Column: COSMOSIL 5C18-AR-II (inner: 4.6 mm×25 cm);

Mobile phase: Acetonitrile (100%): Phosphoric acid (0.085%)=30:70;

Flow rate: 1 ml/min;

Retention time of Flazin is about 12.5 mins.

The UV/VIS absorption spectra of Flazin is 196.1 nm, 290.4 nm and 362.4 nm.

In some embodiments, the composition to be monitored is a fermentation product, which may be purified or unpurified; the fermentation product can be highly purified, or only slightly purified, such as by using liquid-solid separation. In some embodiments, before the step (b) of measuring the amount of the beta-carboline alkaloid compound in a batch of the composition to be monitored, the composition to be monitored is diluted with water.

Without further elaboration, it is believed that one skilled in the art can, based on the disclosure herein, utilize the present invention to its fullest extent.

The following specific examples are, therefore, to be construed as merely descriptive, and not limitative of the disclosure in any way whatsoever.

### EXAMPLES

### Example 1

### Xanthine Oxidase Inhibitory Activity of Flazin

Xanthine oxidase inhibitory activity was measured as follows. Firstly, concentrations (0.625, 1.25, 2.5, 5.0, 10.0 and 20.0 mg/ml) of Flazin samples were prepared. Then, xanthine oxidase inhibitory activity was measured by HPLC by the following procedure. In a reaction tube, 880 μl of xanthine (50 μg/ml in 100 mM PBS) and 40 μl of 50 mM PBS or 40 μl of the Flazin samples were premixed, and 80 μl io of xanthine oxidase (0.1 U) was added to initiate the reaction. The reaction was incubated at 30° C. for 30 min., after which an equal volume of absolute ethanol was added to terminate the reaction. The terminated reaction was filtered through a 0.22 μm membrane filter and the content of xanthine in the reactions was analyzed by HPLC. Xanthine oxidase inhibitory activity of the samples was calculated as follows:

$$X O I ( \% ) = 100 \times \frac{{[ xanthine ]}_{after sample} - {[ xanthine ]}_{after control}}{{[ xanthine ]}_{initial} - {[ xanthine ]}_{after control}}$$

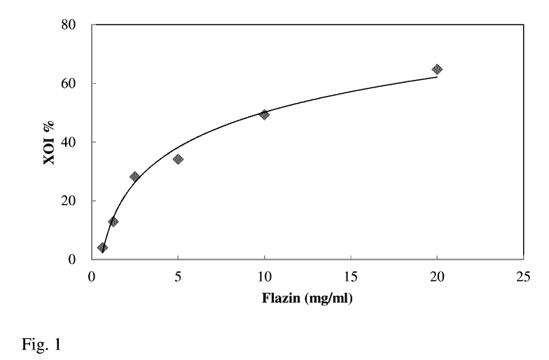

The results are shown in FIG. 1. The IC50 of Flazin was about 9.91 mg/ml.

### Example 2

Two Flazin test samples were prepared (samples A and B), and the concentration of Flazin in samples A and B were determined to be 6.42 ppm and 11.06 ppm, respectively. The xanthine oxidase inhibitory activity of samples A and B was then measured by the method of Example 1. The results show that the XOI activities of samples A and B were 51% and 75%, respectively. The results show that the higher the Flazin concentration in the sample, the higher the XOI activity the sample possesses.

### Example 3

Two additional Flazin test samples were prepared (samples C and D), and the amount of Flazin in samples C and D were measured by HPLC analysis. The results show that the concentration of Flazin in samples C and D was 6.42 ppm and 7.05 ppm, respectively. The xanthine oxidase inhibitory activity of samples C and D was then measured by the method of Example 1. The results show that the xanthine oxidase inhibitory activities of samples C and D was 51% and 56%, respectively. The results show that the higher the Flazin concentration in the sample, the higher the XOI activity the sample possesses.

### Example 4

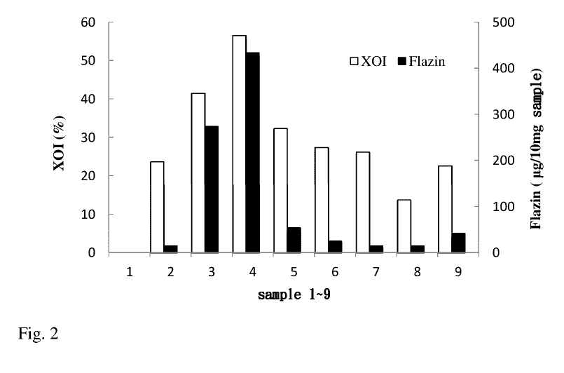

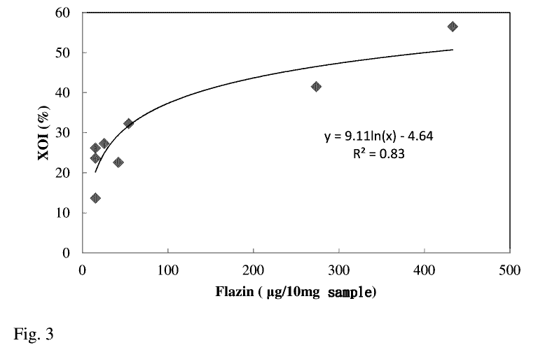

9 flazin samples, having different XOI activities, were analyzed to measure their Flazin concentrations and their xanthine oxidase inhibitory activities. The results are shown in FIG. 2. Sample no. 4 shows the highest Flazin concentration, as well as the highest xanthine oxidase inhibitory activity. Samples with Flazin concentrations lower than 50 ppm exhibit xanthine oxidase inhibitory activities of below 30%. The results show a positive correlation between Flazin concentration (X; ug/10 mg) and the xanthine oxidase inhibitory activity (Y; %). The correlation of Flazin concentration and the xanthine oxidase inhibitory activity are shown in FIG. 3. An exponential equation showing the correlation of Flazin concentration and the xanthine oxidase inhibitory activity was obtained as below.

Y=9.11 ln(X)−4.64, R²=0.8348

The results show that Flazin can be used as an indicator for monitoring XOI activities of samples.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, a person skilled in the art can easily ascertain the essential characteristics of the present invention, and without departing from the spirit and scope thereof, can make various changes and modifications of the present invention to adapt it to various usages and conditions. Thus, other embodiments are also within the claims.

1. A method for inhibiting xanthine oxidase activity in a subject in need thereof, the method comprising contacting a composition comprising a beta-carboline alkaloid compound of formula (I), with the xanthine oxidase, 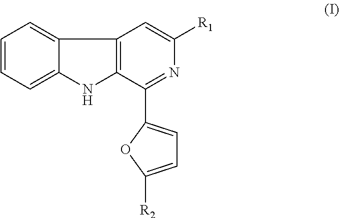
wherein R₁ is selected from the group consisting of a carboxyl group, a carboxylate group, a carboxamide group and hydrogen, and R₂ is selected from the group consisting of —CH₂COOCH₃, a methoxy group, hydrogen and a methyl group.

2. The method of claim 1, wherein R₁ is selected from the group consisting of a carboxyl group and hydrogen.

3. The method of claim 1, wherein R₁ is hydrogen, and R₂ is hydrogen.

4. The method of claim 1, wherein the compound is 1-[5-(hydroxymethyl)furan-2-yl]-9H-pyrido[3,4-b]indole-3-carboxylic acid, and/or 1-(5-hydroxymethyl-2-furyl)-β-carboline-3-carboxylic acid.

5. A method for reducing uric acid levels in a subject in need thereof, the method comprising administering to said subject an effective amount of a beta-carboline alkaloid compound of formula (I) 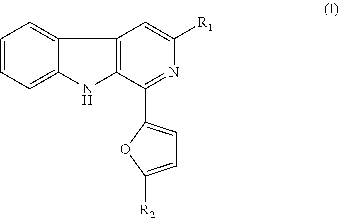
wherein R₁ is selected from the group consisting of a carboxyl group, a carboxylate group, a carboxamide group and hydrogen, R₂ is selected from the group consisting of —CH₂COOCH₃, a methoxy group, hydrogen and a methyl group.

6. The method of claim 5, wherein the subject in need thereof suffers from gout or hyperuricemia.

7. The method of claim 5, wherein the compound is administered orally.

8. The method of claim 5, wherein the compound is in a pure form.

9. A method for monitoring xanthine oxidase inhibition activity, the method comprising:
(a) determining a desirable amount of a beta-carboline alkaloid compound of formula (I), said desirable amount being an amount that correlates with a desirable level of xanthine oxidase inhibition activity, 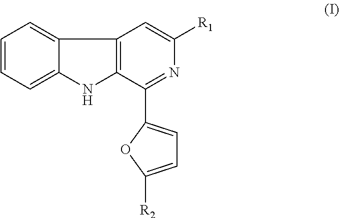
wherein R₁ is selected from the group consisting of a carboxyl group, a carboxylate group, a carboxamide group and hydrogen, and R₂ is selected from the group consisting of —CH₂COOCH₃, a methoxy group, hydrogen and a methyl group;
(b) measuring the amount of said beta-carboline alkaloid compound in a batch of a composition to be monitored; and
(c) determining that said batch has reached said desirable xanthine oxidase inhibition activity when said amount is equal to or higher than said desirable amount.

10. The method of claim 9, wherein R₁ is selected from the group consisting of a carboxyl group and hydrogen.

11. The method of claim 9, wherein R₁ is hydrogen, and R₂ is hydrogen.

12. The method of claim 9, wherein the compound is 1-[5-(hydroxymethyl)furan-2-yl]-9H-pyrido[3,4-b]indole-3-carboxylic acid, and/or 1-(5-hydroxymethyl-2-furyl)-β-carboline-3-carboxylic acid.

13. The method of claim 9, wherein the amount of said beta-carboline alkaloid compound is measured by high performance liquid chromatography (HPLC).

14. The method of claim 9, wherein said composition to be monitored is a fermentation product.

15. The method of claim 9, wherein said composition to be monitored is diluted.
